(19) United States
(12) Patent Application Publication
(10) Pub. No.: US 2007/0166341 A1
(43) Pub. Date: Jul. 19, 2007

(54) Solid volatile composition and method and system for making the same

(76) Inventors: Tetsuo Nakatsu, Pleasantville, NY (US); Ichiro Kubota, Ashikaga (JP)

Correspondence Address: LACKENBACH SIEGEL, LLP, LACKENBACH SIEGEL BUILDING, 1 CHASE ROAD, SCARSDALE, NY, 10583, US

(21) Appl. No.: 11/486,353

(22) Filed: Jul. 12, 2006

Related U.S. Application Data
(60) Provisional application No. 60/698,666, filed on Jul. 12, 2005.

(51) Int. Cl. A61K 8/49; A01N 25/00

(52) U.S. Cl. 424/405; 424/439; 512/1

(57) ABSTRACT

This invention provides a solid air freshener and method for manufacturing the same wherein a composition is constructed from a first material being a solid volatile material at an ambient temperature and a second material being a vicious oily fluid volatile material at ambient temperature. The present invention combines the first and second materials to make a solid, in one embodiment a solid, crystalline or semi-crystalline, air freshener as a substitute for scented candles that is solid at ambient temperatures. The present invention provides for an improved mechanism for delivering scent without a risk of fire or liquid wax. The present product may be retained in a monolithic construct, or ground as a small powder. Applications include a room air freshener, carpet deodorizer, toilet freshener, etc. Antimicrobial oily compositions may be included to limit unhealthy buildups.

### CROSS REFERENCE TO RELATED APPLICATIONS

The present invention claims priority from U.S. Provisional Application Ser. No. 60/698,666 filed Jul. 12, 2005, the entire contents of which are herein incorporated by reference.

### BACKGROUND OF THE INVENTION

1. Field of the Invention

The present invention relates to a solid volatile composition. More specifically, the present invention relates to a solid volatile composition that easily replaces fluid and solvated oils based volatile systems without employing incompatible or non-volatile materials as fillers (e.g. polymers, porous ceramic, and absorbent) and carriers (e.g. inorganic material and waxes), and provides improved performance over liquid volatiles, (e.g. fragrances, perfumes and insect repellents). Even more specifically, the present invention relates to volatile solid composition that is easily portable and usable in a solid, granulated, or paste or gel form without employing incompatible materials as fillers while remaining usable in consumer-based environments including water systems (e.g. bath tab) and highly moisturized systems (e.g. garbage and toilet tanks).

2. Description of the Related Art

Conventional volatile fragrance compositions are commonly liquids, or thermally activated gels. Conventionally, fragrances are provided in a liquid form that is commonly employed in a variety of consumer products including washing detergent, spray air-fresheners, floor cleaners, and many others.

In other conventional embodiments, fragrances are provided in combination with oils and used in conjunction with candles or lamps that raise concerns regarding fires, spills, and dissipate at a very rapid rate.

As a further detriment to employing liquid volatile fragrance compositions around a flame (lamp/candle), dissipation is rapidly increased directly with temperature. Additionally, a common wax candle flame operates in a thermal range of approximately 150-180 C and is therefore of sufficient temperature to chemically deconstruct (breakdown) all liquid volatile fragrances causing rapid volumetric loss, and a substantial rapid decrease in fragrance impact on a relative human sensitivity scale.

In conventional practice, fluid volatile fragrances are frequently difficult to handle and are so volatile they are typically dissolved into another liquid or fluid medium like water for dish soap or alcohol for perfume to enable ready transport.

Also in conventional practice, solid non-volatile matrices have been formed to contain a continuously fluid or oily volatile composition. For example, a pumice stone (a non-volatile solid) has been soaked in an ambient temperature fragrance liquid, and thereafter used as an air freshener. Unfortunately, this conventional art is unsatisfactory and often allows staining and dripping.

Also in conventional practice are fragrances containing solids that dissolve in fluid (for example toilet sanitary packs). Unfortunately, these solids are incapable of containing the solid fragrances of the present invention, and require contact with a fluid to dissolve, preventing their ready use in a toilet tank or bowl.

Fragrance and flavor composition are volatile to carry out an expected effect on olfactory systems and are therefore commonly exist as liquids at room temperature since the relatively small molecules of each component have lower melting points within an ambient temperate range (melt at room temperature). Most of fragrances and flavors formulated into a personal care product (including; personal care product such as perfume, cosmetics, shampoos, and toiletry; room care products such as spray-type air-fresheners and candles, home care products such as washing detergent, cleaner and foods such as candy and many others) act to increase the value and quality, and also the cost of the product. For these consumer-product purposes, liquid fragrances and flavors are more convenient to incorporate into such product even though a liquid form has detriments including complex handling and safety requirements necessitating special container requirements for shipping.

However, a solid form fragrances and flavors are more desirable for applying to products such as air-freshener used in rooms and automobiles, and powdery products such as laundry detergents, carpet cleaners, and dried powdery foods such as tea powders and a flour enabling even mixing. Relating to such a purpose a few conventional technologies have been employed to make a solid form product containing fragrance and flavor, which include synthetic polymers such as polyvinyl chloride (PVC) resin and cyclodextrin; inorganic adsorbent such as silica gel and porous porcelain; inorganic salt such as salt and sodium bicarbonate; waxes such as petroleum wax and bees wax; and even paper and cloth to aid the solidification process.

Unfortunately, these materials individually or in combination have serious drawbacks for delivery of volatiles because of substantial physical and chemical incompatibility each other. Volatiles are composed of small organic compounds which have less than approximately 500 of molecular weight, and are lipophilic, and are more importantly complex mixtures of organic compounds. Thus volatiles are difficult to work with and provide substantial processing problems.

Adsorbents such as PVC resin, silica gel, paper and cloth may conventionally employed to absorb and hold a few percents of organic compounds, but the adsorption property of these absorbents are different from compound to compound, which means less compatible or weakly adsorbed compounds are quickly dispersed and more compatible or strongly adsorbed compounds remain longer. Therefore such products cannot provide consistency and a commonly expected quality of volatile, fragrance and flavor. In addition, many conventional products are oily, greasy, messy, and stain clothing and skin readily because of incompatibility with the fillers and adsorbents, and therefore and provide less diffusive volatile by losing volatiles after a period of time because absorption and diffusion are contradictive phenomenon.

Cyclodextrin and porous porcelain have a “porous” structures. To wit, Cyclodextrin provides a perimeter chain with an inner opening for holding liquids, unfortunately cyclodextrin holds only compounds having a limited size range (due to the crystalline/molecular structure, and porous porcelain releases unevenly volatiles as if it were a liquid in a pot.

Salts are inorganic materials which are completely incompatible with volatile compositions. Unfortunately, the quality of such organic volatiles is difficult to control in any manner.

Paraffin wax, bees wax, and plant wax are mixtures of highly less polar organic compounds such as long chain hydrocarbon or long chain alkyl esters which are also much less affinitive to most of volatile and rather polar fragrance materials and therefore shield and prevent the volatiles in the inside of wax polymer molecule to disperse into the air. In addition, waxes are very unfriendly and messy in ordinal life environments and have a much lower desirability index or property for most fragrances because of the non-polar property and greasy properties of the same.

More specifically, none of the traditional technologies can provide a powdery or particulate solid volatile fragrance and flavor, and the product is only conventionally available as a gel having much lower surface areas than finer powders having large relative surface area to volume to increase dispersion property of the volatile fragrance and flavor. In addition, except specific cyclodextrin and salt, none of traditional technology can generally be used for foods or where skin contact may occur.

U.S. Pat. No. 6,090,774 to Moscona et al., provides a single phase liquid mixture formed from at least two other normally solid perfumery substances, provides some relative background material and is incorporated fully herein by reference.

As an additional example, reference is made to the use of insect repellants having various fluid active ingredients that are readily applied as fluids but difficult to render in a solid form. Reference is made to “Tick Bite Prevention & The Use of Insect Repellants” by Kirby C. Stafford, PhD, Connecticut Agricultural Experiment Station, dated June 2005, located at www.cases.state.ct.us, the contents of which are incorporated herein by reference wherein an extensive table notes active ingredients employed as insect-repellant fluids for the prevention of tick bites.

In summary, the problems with commercially available fluid fragrances are linked to their fluid and gel structures and requisite compositions having low solid-liquid transition temperatures (T_{C}, commonly less than 0° C.). These problems result in at least the following consequences:
1. Extremely rapid volumetric loss at all temperatures particularly elevated ambient temperatures, making useful life as a function of volume very low.
2. Difficulty and danger in storing and transport.
3. High cost of manufacturing fluid fragrances.
4. Difficulty handling fluid fragrances, including spills, fires, discoloration, inability to position as desired (must fill a volume), cannot be shaped without damage or containment to prevent rapid volumetric loss.
5. Low numbers of commercial adaptations for the public benefit.

### OBJECTS AND SUMMARY OF THE INVENTION

One object of the present invention is to provide a solution to one of the above-noted detriments.

Another aspect of the present invention is to provide a solid volatile composition and method for manufacturing the same.

According to another aspect of the present invention is to provide a solid volatile composition having a solidus transition between approximately 20° C. and 150° C.

Another aspect of the present invention is to provide a volatile composition composed of compatible and affinitive components each forming almost a homogenous composition after mixing and requiring almost no additional or extra solvent material.

According to another aspect of the present invention is to provide a volatile solid composition to enhance fragrance performance in solid phases by utilizing a natural property of chemicals; wherein solid (optionally) single materials tend to let minor components, even more volatile materials than the solid, be expressed as a concentration gradient between the surface of the solid and an inside of the volatile solid material, as the volatile material naturally moves from an inside (non-surface) region e to the surface from where volatiles are dispersed thereafter into the air.

According to another aspect of the present invention is to provide a consumer product that his completely solid and volatile and thereby prevents the leaking and staining concerns raised by the conventional arts.

According to an embodiment of the present invention there is provided a solid volatile composition, comprising, a solid volatile material having low vapor pressure at ambient temperature, having at least one polar functional group, or having only one long alkyl chain and melting point below 150° C.

According to an embodiment of the present invention there is provided a solid volatile composition, comprising, a solid volatile material having low vapor pressure at ambient temperature and an oily volatile material at an ambient temperature, wherein the solid composition has a solid state at ambient temperature, and wherein the solid volatile composition comprises, between 50% to 98% of the solid volatile material, between 2%-50% of the oily material.

According to another embodiment of the present invention, there is provided a solid volatile composition, wherein: the solid volatile composition comprises at least one organic molecule noted in Tables I-V.

According to another embodiment of the present invention, there is provided a solid volatile composition, wherein: the solid volatile composition comprises at least one of group consisting of, an acrylic derivative molecule, a cylohaxane derivative molecule, a benzene derivatives molecule, a cyclopentyl derivatives, a furfural acetone molecule, a dimethyl 4-hydroxy furanone molecule, an ethylmaltol molecule, and a hinokitiol molecule.

According to another embodiment of the present invention, there is provided a volatile solid composition, wherein; the volatile solid composition comprises at least one of a group consisting of organic molecules having less than 500 molecular weight with melting points in the range of 20 to 225° C. at normal pressure; wherein the at least one of the group is an organic molecule selected from those listed Tables I-V, noted above.

The above, and other aspects, features and advantages of the present invention will become apparent from the following description read in conduction with the accompanying claims.

### BRIEF DESCRIPTION OF THE DRAWINGS

FIG. 1 is a graphic representation of weight change for a solid volatile composition (aromacrysty) over time.

### DETAILED DESCRIPTION OF THE PREFERRED EMBODIMENTS

In coping with the problems noted above, the present invention provides a system for manufacturing a solid volatile composition that replaces conventionally known liquid fragrance compositions.

What has not been appreciated in the conventional arts are the substantial commercial benefits available in providing a solid volatile composition, including:
(a) Ready shaping or molding to an attractive finished form for packaging, shipping and use.
(b) Simple containment without a physical package, namely the solid is self supporting.
(c) A solid fragrance supply is self eradicating over time; the fragrance is dependent on a function of time (f(t)) as a function of volume (f(v)), of surface area (f(s)), and of temperature (f(T)) related melting points. Thus, as volume decreases, relative surface area:volume increases. For example, a solid construct may loose 10% of its mass in 10 days and maintain the capacity to release a desirable volume fragrance.
(d) Linking of a fragrance composition to a human sensitivity scale allowing ready and repeatable compositional smell-testing in commercial products.
(e) The ready combination of otherwise difficult-to-combine fluid volatile compounds in a single product.
(f) The removal of all fire and thermal damage by removing any burning candle or burning wick operations while allowing the use of the same as an insect or rodent repellent/attractant or insecticide (as either a solid monolith or flowable powder.2
(g) The ready adaptation to multiple sent-masking scenarios including (a) incorporation in a diaper, adult pad, feminine product, or other absorptive material, or (b) garbage or waste spell masking.
(h) The ready manipulation of the material into a thixotropic or other paste (for tooth paste, application on adherent surfaces such as paper, cardboard, and waste receptacles,),

In one aspect of the present invention, a solid volatile composition is provided that replaces scented candle systems, candle+scented oil systems, and fragrance oil lamps with a rigid volatile product.

According to one aspect of the present invention, a solid volatile composition is formed from (a) a main volatile material that is solid at ambient temperatures (roughly 70-90° F.) and (b) a minor oily or fluid volatile material at ambient temperatures. The main volatile material comprising 50-98 volume percent (%) and/or approximate weight % of the combined solid volatile composition. The minor oily or fluid volatile material comprising approximately 2-50 volume percent (%) and/or approximate weight % of the combined solid volatile composition.

In alternatively desired aspects of the present invention, one or more of the volatile materials is selected from the group noted in the following Tables I-V (noted below), including an acryclic derivative molecule, a cylohaxane derivative molecule, a benzene derivatives molecule, a cyclopentyl derivatives, a furfural acetone molecule, a dimethyl 4-hydroxy furanone molecule, an ethylmaltol molecule, and a hinokitiol molecule.

TABLE I
Acyclic derivatives

| Acyclic molecules | mp | Molecular formula |
|---|---|---|
| Levulinic acid | 37 | C₄H₆O₃ |
| Dodecanoic acid | 44-46 | C₁₂H₂₄O₂ |
| Myrstic acid | 53.9 | C₁₄H₂₈O₂ |
| Palmitic acid | 63 | C₁₆H₃₂O₂ |
| Stearic acid | 69-72 | C₁₈H36O₂ |
| Crotonic acid | 70-72 | C₄H₆O₆ |
| Maleic acid | 137-140 | C₄H₄O₄ |
| Tiglic acid | 65 | C₅H₈O₂ |
| Angelic acid | 45 | C₅H₈O₂ |
| Hydroxystearic acid | 75 | C₁₈H₃₆O₃ |
| Amisol SME (N-2- | 91-95 | C₂₀H₄₁O₂N |
| hydroxyethylaclyamide) | | |
| Amisol CME (N-2- | 67-71 | Mixture of |
| hydroxylacylamide derivative | | RCONHCH₂C |
| from coconut oil) | | H₂OH |
| | | R = Coconyl- |

TABLE II
Cylohaxane derivatives

| Cyclic molecules | mp | Molecular formula |
|---|---|---|
| Menthol | 43-45 | C₁₀H₂₀O |
| Patchouli Alcohol | 56 | C₁₅H₂₆O |
| Cedrol | 86 | C₁₅H₂₆O |
| Cedrenol | 56 | C₁₅H₂₄O |
| Scraleol | 106 | C₂₀H₁₆O₂ |
| Caryophyllene alcohol | 94-96 | C₁₅H₂₆O |
| Caryophyllene oxide | 64 | C₁₅H₂₄O |
| Acetoin dimer | 85 or 95 | C₈H₁₆O₄ |
| Ambroxane | 76 | C₁₆H₂₂O |
| Cyclopentadecanone | 63 | C₁₅H₂₈O |
| Cedryl acetate | 80 | C₁₇H₂₈O₂ |
| Caryophyllene acetate | 40 | C₁₇H₂₈O₂ |
| 3-Thujanol | 67 | C₁₀H₁₈O |
| Acetyl cerdrene | 105-110 | C₁₇H₂₆O |
| Fenchyl alcohol | 45-47 | C₁₀H₁₈O |

TABLE III
Benzene Derivatives

| Benzenoid molecules | mp | Molecular formula |
|---|---|---|
| Vanillyl alcohol | 113-115 | C₈H₁₀O₃ |
| p-Ethylphenol | 42-45 | C₈H₁₀O |
| Thymol | 51.5 | C₁₀H₁₄O |
| Hydroquionone dimethylether | 56-60 | C₈H₁₀O₂ |
| Dimethoxy phenol | 53-56 | C₈H₁₀O₃ |
| Ethyl isoeugenol | 62-64 | C₁₂H₁₆O₂ |
| Benzyl iso-eugenol-t | 57 | C₁₇H₁₈O₂ |
| Methoxy cinnamic aldehyde | 44-45 | C₁₀H₁₀O |
| Vanillin | 83 | C₈H₈O₃ |
| Ethjyl Vanillin | 77-78 | C₉H₁₀O₃ |
| Methyl vanillin | 42-45 | C₉H₁₀O₃ |
| 3,5-Dimethyl-1,2- | 91-92 | C₇H₁₀O₂ |
| cyclopentadione | | |
| 3,4-Dimethyl-1,2- | 66 | C₇H₁₀O₂ |
| cyclopentadione | | |
| p-tert-Butyl cyclohexanone | 47 | C₁₀H₁₈O |
| Heliotropine | 42-45 | C₈H₆O₃ |
| Musk Xylol | 112.5 | C₁₂H₁₅N₃O₅ |
| Vanillyl alcohol | 113-115 | C₈H₁₀O₃ |
| Naphtol methyl ether | 72-73 | C₁₁H₁₀O |
| Hydroquinone dimethyl ether | 56-60 | C₈H₁₀O₂ |
| 2,6-Dimethoxy phenol | 53-56 | C₈H₁₀O₃ |
| Vanitrope | 86-88 | C₁₁H₁₄O₂ |
| Ethylisoeugenol | 62-64 | C₁₂H₁₆O₂ |
| Benzyl-iso-eugenol | 57 | C₁₇H₁₈O₂ |
| Raspberry ketone | 82-83 | C₁₀H₁₂O₂ |
| Perillatine | 102 | C₁₀H₁₅NO |
| Phenyl acetoaldehyde trimer | 115 | C₂₄H₂₄O₃ |
| Rose phenone | 86-89 | C₁₀H₉O₂Cl₃ |
| Acetyl iso-eugenol | 80 | C₁₂H₁₄O₃ |
| Diacetyl trimer | 105 | C₁₂H₁₈O₆ |
| Methylanisate | 49-51 | C₉H₁₀O₃ |
| Cresyl phenylacetate | 74-75 | C₁₅H₁₄O₂ |
| Cinnamyl cinnamate | 45 | C₁₈H₁₆O₂ |
| Phenyl ethyl cinnamate | 58 | C₁₇H₁₆O₂ |
| Phenyl salicylate | 44-46 | C₁₃H₁₀O₃ |
| Phenyl ethyl salicylate | 44-46 | C₁₅H₁₄O₃ |
| Methyl atrarate | 143 | C₁₀H₁₂O₄ |
| Phenyl ethyl anthranilate | 42 | C₁₅H₁₅O₂N |
| Cinnamyl anthranilate | 64 | C₁₆H₁₅O₂N |
| Methyl nicotinate | 42-43 | C₇H₇O₂N |
| Indole | 52-54 | C₈H₇N |
| Methoxy cinnamic aldehyde | 44-46 | C₁₀H₁₀O₂ |
| 1,4-Dimethoxy benzene | 54-56 | C₈H₁₀O₂ |
| Calone | 38-39 | C₁₀H₁₀O₃ |
| Diosphenol | 83 | C₁₀H₁₀O₂ |
| Anisylidene acetone | 74 | C₁₁H₁₂O₂ |
| Zingerone | 41 | C₁₁H₁₄O₃ |
| Heliotropyl acetone | 53 | C₁₁H₁₂O₃ |
| Methyl anisal acetone | 60 | C₁₂H₁₄O₂ |
| Methyl naphtyl ketone | 53-55 | C₁₂H₁₀O |
| Benzo-phenone | 49-51 | C₁₃H₁₀O |
| Musk xtlol | 112 | C₁₂H₁₅N₃O₆ |
| Musk ambrette | 83 | C₁₂H₁₆N₂O₅ |
| Musk tibetene | 136 | C₁₃H₁₈N₂O₄ |
| Moskene | 132 | C₁₄H₁₈N₂O₄ |
| Celestlide | 77-78 | C₁₇H₂₄O |
| Versalide | 43 | C₁₈H₂₆O |
| Tonalid | 46 | C₁₈H₂₆O |
| Benzoic acid | 122-123 | C₇H₆O₂ |
| Phenyl acetic acid | 77-78.5 | C₈H₈O₂ |
| Cinnamic acid | 133-134 | C₉H₈O₂ |
| Phenyl prpionic acid | 47-49 | C₈H₁₀O₂ |
| Vanillic acid | 210-213 | C₈H₈O₄ |
| Courmarin | 68.5-69.5 | C₉H₆O₂ |
| Mehyl courmarin | 75 | C₁₀H₈O₂ |
| Piperony acetate | 51 | C₁₀H₁₀O₄ |
| Acetyl vanillin | 77-79 | C₁₀H₁₀O₄ |
| Skatole | 95-96 | C₉H₉N |
| Piperine | 131-135 | C₁₇H₁₉O₃N |
| Capsaicin | 62-65 | C₁₈H₂₇O₃N |
| Nonanoyl vanilly amide | | C₁₇H₂₇O₃N |
| Hydroxy styrene -p | 68-69 | C₈H₈O |
| Hydroxyphenyl acetic acid p - | 148 | C₈H₈O₃ |
| Hydroxyphenyl acetic acid | 129 | C₈H₈O₃ |
| m - | | |
| Hydroxyphenyl propionic | 82-83 | C₁₉H₁₀O₃ |
| acid- o- | | |
| Mint sulfide | 64 | C₁₅H₂₂S |
| Thialdine | 47 | C₆H₁₃NS₂ |
| Ethyl-4-hydroxybenzoate | 114-117 | C₉H₁₀O₃ |
| BHT (Butyl | 70-73 | C₁₅H₂₄O |
| BHA | 48-55 | C₁₁H₁₆O₂ |
| 3,5-di-t-butyl-phenol | 94-95 | C₁₄H₂₂O |

TABLE IV
Cyclopentyl derivatives

| | | Molecular |
|---|---|---|
| Cyclopentyl type molecules | mp | formula |
| Furyl acrolein | 48-50 | C₇H₆O₂ |
| Cyclotene | 105-107 | C₆H₈O₂ |
| Furyl acrolein | 48-50 | C₇H₆O₂ |

TABLE V
Other derivatives

| | | Molecular |
|---|---|---|
| Other type molecules | mp | formula |
| Furfural acetone | 40 | C₄H₈O₂ |
| Dimethyl 4-hydroxy furanone | 78-83 | C₆H₈O₃ |
| Ethylmaltol | 89-91 | C₇H₈O₃ |
| Hinokitiol | 48-52.5 | C₁₀H₁₂O₂ |

All materials may be obtained commercially, for example from the Kawaken Fine Chemical Co., Ltd. of Tokyo Japan.

In select preferred aspects the volatile materials include ethyl vanillin, vanillin, menthol, cinnamic acid, and musk keytone.

In other preferred aspects the solid volatile composition is preferably 70 to 90% of the main solid volatile material.

In other preferred aspects, the oily volatile material is selected from one of a fragrance composition, an insect composition, and antimicrobial composition, and an emotionally stimulating composition like peppermint.

Referring now to a manufacturing method for the above-discussed solid volatile composition. In a first step a desired solid volatile material is selected from the group noted above having a melting point (Tc) within a functionally suitable range, namely above 90° F. (maximum ambient temperature), and a desired oily volatile material as noted above.

In a second step, the solid volatile material is brought to a liquidification temperature and combined by stirring with the oily or fluid volatile material. In a third step, the combined liquid is allowed to cool to ambient temperatures and consequently converts to a solid form that may be processed into alternative sizes, shapes, powders, pastes, etc.

Alternative manufacturing steps including pouring the hot combination into a mold of a desired functional shape and allowing the mold to cool.

Another alternative manufacturing step involves forming the solid volatile composition alone without combination with an oily fluid volatile composition, and thereafter soaking the solid volatile composition in the oily fluid composition allowing absorption through porosity and surface defects.

As discussed above a solid volatile composition as substantial product benefits to the consuming public. These include the ready simple formation of the composition into a candle-shaped replacement for easy integration with existing candle holders and candle apparatus. Also enabled are ready formation of the novelty shapes and other commercially viable shapes. Further, the present solid volatile composition does not require contact with an external fluid (water) to dissolve, ultimately the present invention will dissipate on its own.

In a further aspect of the present invention, the discussion solid volatile composition may be operated as a candle element adjacent a wick, whereby during an operation of a wick, localized ambient temperatures are increased rapidly transitioning the present solid composition to a vapor for immediate volumetric changes in fragrance delivery.

Alternative aspects of the present invention are discussed below:

### Embodiment: 1

Commercial menthol having a melting point in the range of 43 to 45° C. was weighed and the oil was added. The mixture was heated to give clear solution. The solution was poured into the egg shaped mold and cooled to volatile solid composition. The composition was set in a limited volume room (a bath room) for evaluation and measurement of weight decrease.

| | Description |
|---|---|
| Term | Ratio (%) by weight |
| Solid small organic material | |
| Menthol | 85 |
| Oily/organic material | |
| AS-Melon | 15 |
| Form | Solid egg-like solid |
| Order property | Fresh, green melon |

AS-Melon: 5% Musk T; 35% Verdox; 5% Prenyl acetate; 5% Lilial: 50% Fruit base

Evaluation:

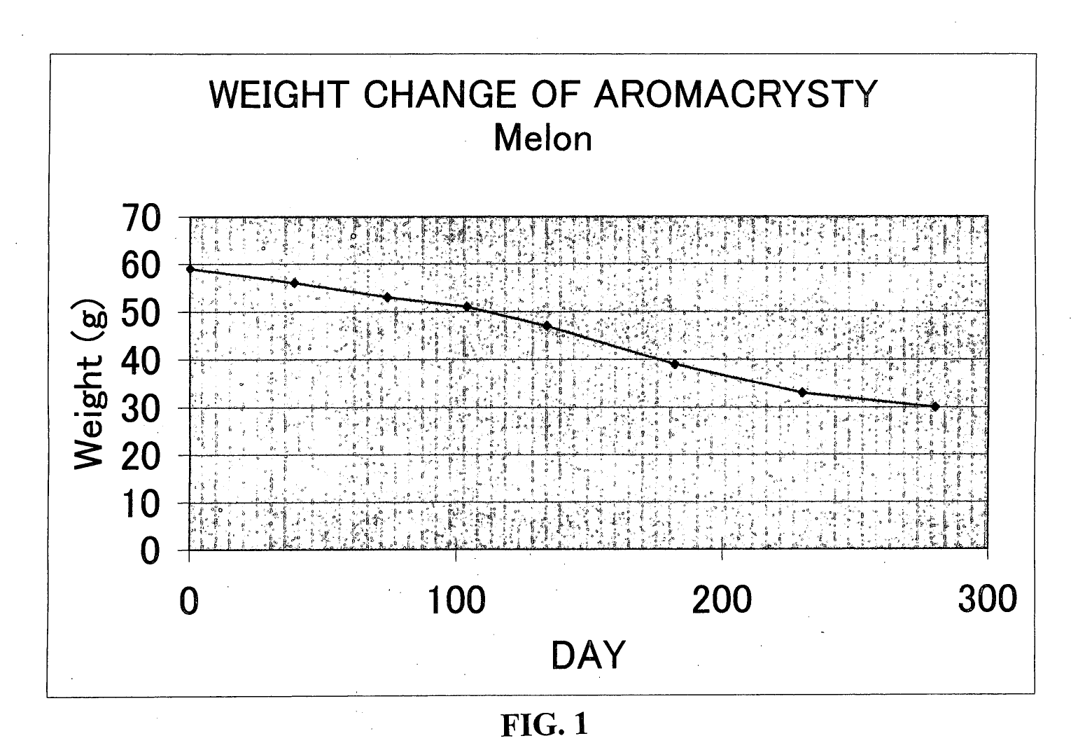

Referring now to FIG. 1, the odor property of the composition was monitored by weighing the specimen over time (See Table VI below) the solid composition was not over powering for long period of time and whenever the door was opened, the pleasant odor was recognizable for the moment.

TABLE VI
Weight change of Crystal over time.

| Day | Weight of composition (g) |
|---|---|
| 0 | 59 |
| 39 | 56 |
| 74 | 53 |
| 104 | 51 |
| 134 | 47 |
| 182 | 39 |
| 230 | 33 |
| 280 | 30 |
| 318 | 28 |

### Embodiment 2

Commercial menthol having a melting point in the range of 43 to 45° C. was weighed and oil/fluid organic was added. The mixture was heated to give a clear solution was cooled to provide a volatile solid composition (VSC).

| | Description |
|---|---|
| Term | Ratio (%) |
| Solid small organic material | |
| Menthol | 90 |
| Oily material | |
| AS-Strawberry* | 10 |
| Form | Needle crystal-like solid |
| Odor property | Fresh, sweet strawberry, very diffusive |
| Pleasant odor index | 90 |
| Camphor-like odor index | 5 |

*AS-Strawberry: 70% of Hexadecanal, 8% of Ethyl butylate, 8% of Methylanthranilate, 4% of Methyl anisate; 4% of Undecalactone, 6% of fruity base

### Embodiment 3

Commercial stearic acid having a melting point in the range of 69 to 72° C. was weighed and the oil was added. The mixture was heated to give clear solution. The solution was poured into the mold and cooled to volatile solid composition (SVC). The composition was evaluated for quenching urine odor. Human urine was mixed with the solid material and the solid volatile composition. A control used was a mixture of urine and water.

| | Description |
|---|---|
| Term | Ratio (%) by weight |
| Solid small organic material | |
| Stearic acid | 80 |
| Oily material | |
| AS-Mint | 20 |
| Form | Solid plate |
| Odor property - based on a | Fresh minty and green |
| defined human odor index. | |

| | Control (Urine + | Base (Urine + | Test sample (Urine + |
|---|---|---|---|
| | Water) | Stearic acid) | Mint composition) |
| Evaluation | Very strong | Strong urine | Almost no urine |
| | unpleasant urine | odor | odor with fresh |
| | and ammonium | | minty note |
| | odor | | |

### Embodiment 4

Commercial stearic acid having a melting point in the range of 69 to 72° C. and benzoic acid having a melting point 122-123° C. were weighed and the oil was added. Benzoic acid was mixed to increase solubility in water. The mixture was heated to give a clear solution and the solution was poured into a mold and cooled to form a volatile solid composition (SVC). The composition was evaluated for solubility in toiletry use (in a toilet or bath).

| | Description |
|---|---|
| Term | Ratio (%) by weight |
| Solid small organic material | |
| Stearic acid | 60 |
| Benzoic acid | 30 |
| Oily material | |
| AS-Mint | 10 |
| Form | Solid plate |
| Odor property | Fresh minty and green |

Result: The weight of the composition decreased 1% by 3-4 times use and flashing everyday.

### Embodiment 5

Commercial stearic acid having a melting point in the range of 69 to 72° C. was weighed and the commercial perfume was added. The mixture was heated to give a clear solution, and was poured into a mold and cooled to volatile solid composition. The composition was applied onto a user's skin, and evaluated.

| | Description |
|---|---|
| Term | Ratio (%) by weight |
| Solid small organic material | |
| Stearic acid | 90 |
| Oily material | |
| Polo sport | 10 |
| Form | Solid plate |
| Odor property | Polo perfume with the original character |

Evaluation:

No greasy feeling and no alcohol odor were uncovered. A scent was very pleasant with an original perfume note. The odor was retained over a few hours with same character (no decrease in the odor quality). As an aside it was reported that this composition kept almost the same odor character over a month.

### Embodiment 6

Commercial ethylvanillin having a melting point of 77° C. was weighed and the oil/organic fluid was added. The mixture was heated to give clear solution. The solution was cooled to provide a volatile solid composition (SVC).

| Term | Description |
|---|---|
| Solid small organic material | |
| Ethylvanillin | 85% |
| Oily material | |
| AS-Garden* | 15% |
| Odor property | Lavender sweet weak vanilla like odor |

*AS-Garden: 18% of ethylenebrassilate; 8% of Iso E Super, 4% of hedione, 15% of linalool, 20% of linalyl acetate, 20% of lavender oil, 4% of rosemary oil, 11% of floral base.

### Embodiment 7

Commercial ethylmaltol having a melting point of 89° C.-91° C. was weighed and a commercial essential amount was added. The mixture was heated to give a clear solution an the solution itself was cooled to provide a volatile solid composition (SVC).

| Term | Description |
|---|---|
| Solid small organic material | |
| Ethylmaltol | 95% |
| Oily material | |
| Natural grapefruit oil | 5 |
| Odor property | Fresh, natural, sweet grapefruit like odor |

Additional applications are further discussed below, involving antibacterial, anti-urinary calculus, anti-fungal, anti-inflammatory, and other aspects all related to the SVC composition family. Several of these applications are noted in detail.

Alternative embodiments are envisioned, including the following:
(1) The use of the enclosed solid volatile composition as a granular or solid particulate in a personal hygiene product such as a baby diaper, adult moisture control pad, feminine hygiene product and wound care pad, wherein the SVC is combined with padding or stuffing during manufacturer.
(2) The use in combination with a water system, wherein the solid volatile composition may be employed in a toilet or other water system to release fragrance and control microorganisms causing malodor, and otherwise kill or prohibit the reproduction of misquote and other insect life.
(3) The use of the SVC in combination (in powder form) with an insecticide such as DEET or Picaridin, wherein the presently formed structure will retain the oily material (DEED or Picardin) for a lasting period without dispersion in rain or water, thereby increasing insect prohibition.
(4) The use of SVC's in combination with other water soluble materials such as N-lauroyl sacrosinate which may be used to foam the material.
(5) The use of SVC's as toot paste (when mixed with water) for personal hygiene assistance to those unable to otherwise avail themselves of common toothbrush/toothpaste.
(6) The use of SVC's as an insect repellant or insecticide when in combination with DEED, pyrethroids, IR3535®, picaridins, p-methane-3,8 diol, eucalyptus oil, citronella oil, and botanical repellants, and the use of the same as a bait or toxic poison in an insect or rodent traps.

In sum, the present invention responds to at least one of the detriments and consequential needs noted above, including easy handling and transportation, simplified packaging and use, easy application of a non-existing product, ready adaptation of a simple system for almost every fragrance creation as no solvent is needed to transport a fragrance, and a comprehensive solid product allowing even and equal delivery of a desired fragrance.

Having described at least one of the preferred embodiments of the present invention with reference to the accompanying drawings, it is to be understood that the invention is not limited to those precise aspects, and that various changes, modifications, and adaptations may be effected therein by one skilled in the art without departing from the scope or spirit of the invention as defined in the appended claims.

What is claimed is:

1. A solid volatile composition, comprising:
a solid small volatile material at ambient temperature and an oily-fluid volatile material at an ambient temperature, wherein said solid volatile composition has a solid state at said ambient temperature, and wherein said solid volatile composition (SVC) comprises:
between 2%-50% of said oily-fluid material; and
between 50% to 98% of said solid small volatile material, wherein said solid small volatile material is selected as any one of the materials in Tables, VII-XI.
TABLE VII
Acyclic derivatives

| | | Molecular |
|---|---|---|
| Acyclic molecules | mp | formula |
| Levulinic acid | 37 | C₄H₆O₃ |
| Dodecanoic acid | 44-46 | C₁₂H₂₄O₂ |
| Myrstic acid | 53.9 | C₁₄H₂₈O₂ |
| Palmitic acid | 63 | C₁₆H₃₂O₂ |
| Stearic acid | 69-72 | C₁₈H36O₂ |
| Crotonic acid | 70-72 | C₄H₆O₆ |
| Maleic acid | 137-140 | C₄H₄O₄ |
| Tiglic acid | 65 | C₅H₈O₂ |
| Angelic acid | 45 | C₅H₈O₂ |
| Hydroxystearic acid | 75 | C₁₈H₃₆O₃ |
| Amisol SME (N-2- | 91-95 | C₂₀H₄₁O₂N |
| hydroxyethylaclyamide) | | |
| Amisol CME (N-2- | 67-71 | Mixture of |
| hydroxylacylamide derivative | | RCONHCH₂CH₂OH |
| from coconut oil) | | R = Coconyl- |

TABLE VIII
Cylohaxane derivatives

| | | Molecular |
|---|---|---|
| Cyclic molecules | mp | formula |
| Menthol | 43-45 | C₁₀H₂₀O |
| Patchouli Alcohol | 56 | C₁₅H₂₆O |
| Cedrol | 86 | C₁₅H₂₆O |
| Cedrenol | 56 | C₁₅H₂₄O |
| Scraleol | 106 | C₂₀H₁₆O₂ |
| Caryophyllene alcohol | 94-96 | C₁₅H₂₆O |
| Caryophyllene oxide | 64 | C₁₅H₂₄O |
| Acetoin dimer | 85 or 95 | C₈H₁₆O₄ |
| Ambroxane | 76 | C₁₆H₂₂O |
| Cyclopentadecanone | 63 | C₁₅H₂₈O |
| Cedryl acetate | 80 | C₁₇H₂₈O₂ |
| Caryophyllene acetate | 40 | C₁₇H₂₈O₂ |
| 3-Thujanol | 67 | C₁₀H₁₈O |
| Acetyl cerdrene | 105-110 | C₁₇H₂₆O |
| Fenchyl alcohol | 45-47 | C₁₀H₁₈O |

TABLE IX
Benzene Derivatives

| | | Molecular |
|---|---|---|
| Benzenoid molecules | mp | formula |
| Vanillyl alcohol | 113-115 | C₈H₁₀O₃ |
| p-Ethylphenol | 42-45 | C₈H₁₀O |
| Thymol | 51.5 | C₁₀H₁₄O |
| Hydroquionone dimethylether | 56-60 | C₈H₁₀O₂ |
| Dimethoxy phenol | 53-56 | C₈H₁₀O₃ |
| Ethyl isoeugenol | 62-64 | C₁₂H₁₆O₂ |
| Benzyl iso-eugenol-t | 57 | C₁₇H₁₈O₂ |
| Methoxy cinnamic aldehyde | 44-45 | C₁₀H₁₀O |
| Vanillin | 83 | C₈H₈O₃ |
| Ethjyl Vanillin | 77-78 | C₉H₁₀O₃ |
| Methyl vanillin | 42-45 | C₉H₁₀O₃ |
| 3,5-Dimethyl-1,2- | 91-92 | C₇H₁₀O₂ |
| cyclopentadione | | |
| 3,4-Dimethyl-1,2 | 66 | C₇H₁₀O₂ |
| cyclopentadione | | |
| p-tert-Butyl cyclohexanone | 47 | C₁₀H₁₈O |
| Heliotropine | 42-45 | C₈H₆O₃ |
| Musk Xylol | 112.5 | C₁₂H₁₅N₃O₅ |
| Vanillyl alcohol | 113-115 | C₈H₁₀O₃ |
| Naphtol methyl ether | 72-73 | C₁₁H₁₀O |
| Hydroquinone dimethyl ether | 56-60 | C₈H₁₀O₂ |
| 2,6-Dimethoxy phenol | 53-56 | C₈H₁₀O₃ |
| Vanitrope | 86-88 | C₁₁H₁₄O₂ |
| Ethylisoeugenol | 62-64 | C₁₂H₁₆O₂ |
| Benzyl-iso-eugenol | 57 | C₁₇H₁₈O₂ |
| Raspberry ketone | 82-83 | C₁₀H₁₂O₂ |
| Perillatine | 102 | C₁₀H₁₅NO |
| Phenyl acetoaldehyde trimer | 115 | C₂₄H₂₄O₃ |
| Rose phenone | 86-89 | C₁₀H₉O₂Cl₃ |
| Acetyl iso-eugenol | 80 | C₁₂H₁₄O₃ |
| Diacetyl trimer | 105 | C₁₂H₁₈O₆ |
| Methylanisate | 49-51 | C₉H₁₀O₃ |
| Cresyl phenylacetate | 74-75 | C₁₅H₁₄O₂ |
| Cinnamyl cinnamate | 45 | C₁₈H₁₆O₂ |
| Phenyl ethyl cinnamate | 58 | C₁₇H₁₆O₂ |
| Phenyl salicylate | 44-46 | C₁₃H₁₀O₃ |
| Phenyl ethyl salicylate | 44-46 | C₁₅H₁₄O₃ |
| Methyl atrarate | 143 | C₁₀H₁₂O₄ |
| Phenyl ethyl anthranilate | 42 | C₁₅H₁₅O₂N |
| Cinnamyl anthranilate | 64 | C₁₆H₁₅O₂N |
| Methyl nicotinate | 42-43 | C₇H₇O₂N |
| Indole | 52-54 | C₈H₇N |
| Methoxy cinnamic aldehyde | 44-46 | C₁₀H₁₀O₂ |
| 1,4-Dimethoxy benzene | 54-56 | C₈H₁₀O₂ |
| Calone | 38-39 | C₁₀H₁₀O₃ |
| Diosphenol | 83 | C₁₀H₁₀O₂ |
| Anisylidene acetone | 74 | C₁₁H₁₂O₂ |
| Zingerone | 41 | C₁₁H₁₄O₃ |
| Heliotropyl acetone | 53 | C₁₁H₁₂O₃ |
| Methyl anisal acetone | 60 | C₁₂H₁₄O₂ |
| Methyl naphtyl ketone | 53-55 | C₁₂H₁₀O |
| Benzo-phenone | 49-51 | C₁₃H₁₀O |
| Musk xtlol | 112 | C₁₂H₁₅N₃O₆ |
| Musk ambrette | 83 | C₁₂H₁₆N₂O₅ |
| Musk tibetene | 136 | C₁₃H₁₈N₂O₄ |
| Moskene | 132 | C₁₄H₁₈N₂O₄ |
| Celestlide | 77-78 | C₁₇H₂₄O |
| Versalide | 43 | C₁₈H₂₆O |
| Tonalid | 46 | C₁₈H₂₆O |
| Benzoic acid | 122-123 | C₇H₆O₂ |
| Phenyl acetic acid | 77-78.5 | C₈H₈O₂ |
| Cinnamic acid | 133-134 | C₉H₈O₂ |
| Phenyl prpionic acid | 47-49 | C₈H₁₀O₂ |
| Vanillic acid | 210-213 | C₈H₈O₄ |
| Courmarin | 68.5-69.5 | C₉H₆O₂ |
| Mehyl courmarin | 75 | C₁₀H₈O₂ |
| Piperony acetate | 51 | C₁₀H₁₀O₄ |
| Acetyl vanillin | 77-79 | C₁₀H₁₀O₄ |
| Skatole | 95-96 | C₉H₉N |
| Piperine | 131-135 | C₁₇H₁₉O₃N |
| Capsaicin | 62-65 | C₁₈H₂₇O₃N |
| Nonanoyl vanilly amide | | C₁₇H₂₇O₃N |
| Hydroxy styrene-p | 68-69 | C₈H₈O |
| Hydroxyphenyl acetic acid p - | 148 | C₈H₈O₃ |
| Hydroxyphenyl acetic acid m - | 129 | C₈H₈O₃ |
| Hydroxyphenyl propionic acid- o- | 82-83 | C₁₉H₁₀O₃ |
| Mint sulfide | 64 | C₁₅H₂₂S |
| Thialdine | 47 | C₆H₁₃NS₂ |
| Ethyl-4-hydroxybenzoate | 114-117 | C₉H₁₀O₃ |
| BHT (Butyl | 70-73 | C₁₅H₂₄O |
| BHA | 48-55 | C₁₁H₁₆O₂ |
| 3,5-di-t-butyl-phenol | 94-95 | C₁₄H₂₂O |

TABLE X
Cyclopentyl derivatives

| | | Molecular |
|---|---|---|
| Cyclopentyl type molecules | mp | formula |
| Furyl acrolein | 48-50 | C₇H₆O₂ |
| Cyclotene | 105-107 | C₆H₈O₂ |
| Furyl acrolein | 48-50 | C₇H₆O₂ |

TABLE XI
Other derivatives

| | | Molecular |
|---|---|---|
| Other type molecules | mp | formula |
| Furfural acetone | 40 | C₄H₈O₂ |
| Dimethyl 4-hydroxy furanone | 78-83 | C₆H₈O₃ |
| Ethylmaltol | 89-91 | C₇H₈O₃ |
| Hinokitiol | 48-52.5 | C₁₀H₁₂O₂ |

2. A volatile solid composition, according to claim 1, wherein
the solid small organic material and the oily volatile material are homogeneous upon heating.

3. A solid volatile composition, according to claim 1, wherein:
the composition preferably comprises between approximately 70% to 95% of the solid volatile materials.

4. A solid volatile composition, according to claim 1, wherein:
the composition preferably comprises at least one material selected from a solid volatile group consisting of menthol, ethyl vanillin, vanillin, ethylvanillin, ethylmaltol, and maltol.

6. A solid volatile composition, according to claim 1, wherein:
the volatile oily material is a fragrance composition.

7. A solid volatile composition, according to claim 1, wherein:
the volatile oily mixture is an insect repellant.

8. A solid volatile composition, according to claim 1, wherein:
the volatile oily material is a favor composition.

9. A solid volatile composition, according to claim 1, wherein:
the volatile oily materials comprise an antimicrobial composition.

10. A solid volatile composition, according to claim 1, wherein:
the volatile composition preferably has a transition temperature (T_{C}) of between 33° C. and 120° C. and preferably 90° C.

11. A solid volatile composition, according to claim 8, wherein:
the volatile composition preferably has a transition temperature (T_{C}) of between 50° C. and 120° C., and preferably 90° C.

12. A solid volatile composition, according to claim 8, wherein:
the volatile composition preferably has a transition temperature (T_{C}) of between 65° C. and 120 C, and preferably 90° C.

13. A solid volatile composition, according to claim 8, wherein:
the volatile composition preferably has a transition temperature (T_{C}) of between 70° C. and 120° C., and preferably 85° C.

14. A volatile solid composition, according to claim 1, wherein:
the volatile solid composition in combination with at least one of the following, a powder detergent, a talcum powder, a flower, a bean flour, a condiment, a fertilizer, a powdery insect repellent, a carpet cleaner, a floor cleaner, garbage odor neutralizer, an absorptive personal hygiene product.
